(19) United States
(12) Patent Application Publication
(10) Pub. No.: US 2020/0324005 A1
(43) Pub. Date: Oct. 15, 2020

(54) Apparatus and Method for the Sterilization of Airport Security Scanner Bins

(71) Applicant: Ronald Scott Frazier, Tamuning, GU (US)

(72) Inventor: Ronald Scott Frazier, Tamuning, GU (US)

(21) Appl. No.: 16/843,566

(22) Filed: Apr. 8, 2020

Related U.S. Application Data
(60) Provisional application No. 62/819,592, filed on Apr. 15, 2019.

(51) Int. Cl. A61L 2/10; A61L 2/26

(52) U.S. Cl. CPC A61L 2/10; A61L 2/26; A61L 2202/23; A61L 2202/122; A61L 2202/11

(57) ABSTRACT

An apparatus for sterilizing airport security scanner bins includes a housing having a housing bottom support, housing sidewalls, and a housing top wall, the housing having an open front housing end and an open back housing end. The apparatus includes a top UV radiator, and side UV radiators within the housing and front and back UV radiators. The housing and the open front and back ends are sized to allow an airport security scanner bin to pass through the housing while being radiated with UV light on a top, bottom, front wall, back wall and sidewalls of the bin. The housing bottom support can be a conveyor.

This application claims the benefit of U.S. Provisional Application 62/819,592 filed Apr. 15, 2019.

### BACK GROUND

The present inventor has recognized that airport security scanner bins used during the cheek-in process may contain amounts of bacteria or viral contaminates. This bacterium or viral contaminates are a result of cross contamination by sick passengers, passenger shoes, airport workers working in or passing through the security area.

The present inventor has recognized that it would be desirable to sterilize the airport security scanner bins and to protect the traveling passengers and personnel from possible viral or bacterial contamination.

### SUMMARY

An exemplary apparatus for sterilizing airport security scanner bins, includes a housing having a housing bottom support, housing sidewalls, and a housing top wall, the housing having an open front housing end and an open back housing end. The apparatus includes a top ultraviolet or “UV” radiator, side UV radiators within the housing, and front and back UV radiators. The housing and the open front and back ends are sized to allow an airport security scanner bin to pass through the housing while being radiated with UV light on a top, bottom, front wall, back wall and sidewalls of the bin.

The housing bottom support can comprise a conveyor.

The front UV radiator is located adjacent the open front housing end and the back UV radiator is located adjacent to the open back housing end.

The apparatus can include a bin stacking table for receiving bins from the open back housing end.

The side UV radiators can comprise ultraviolet C (wavelength 200-280 nm) or “UVC” lights or UV LED diode arrays. The top UV radiator can comprise a UVC LED diode array. The front and back UV can comprise UVC LED diode arrays.

The side UV radiators can have a length substantially equal to a length of the housing. The top UV radiator can have a length substantially equal to a length of the housing. The front and back UV radiators can have a length substantially equal to a width of the housing.

The apparatus can include a bin sensor for sensing that a bin is in position within the housing to turn on the UV radiators for a pre-selected period of time.

An exemplary embodiment of the present invention is a security scanner bin sterilizer apparatus. Advantageously, the apparatus is controlled by a programmable logic controller with a human machine interface (PLC/HMI) that operates automatically or can be operated by manual control. This apparatus utilizes UVC Lights, which can radiate ultraviolet radiation at wavelengths less than 300 nm, or UVC LED diode arrays, which can radiate UV light in a wavelength range of 250 nm to 279 nm, that are strategically positioned to radiate the needed areas of the security bins for sterilization. A wavelength of 274 nm is effective. The machine has power supplies to provide, the UVC diode arrays or

UVC lights, conveyors, stacker and the PLC/HMI, the necessary operating voltages. The machine has three conveyor belts to move the security scanner bins into position for sterilization. The first and second conveyors operate at a preset speed while the third conveyor operates at a faster speed for stacking purposes. Once the security scanner bin reaches the sensor, the conveyors stop, and the sterilization process activates.

This sterilization process, radiating of UVC light, operates for a programmed duration, between 3.5 to 5 seconds. One of the front or back UVC radiators, or both, can be operated while the bin is moving into or out of the housing to sterilize a bottom of the bin. The top UVC radiator and the side UVC radiators could be operated while the bin is moving through the housing as well.

The last, third conveyor moves the bin to a stacking mechanism, which stacks the bins for redistribution by the operator.

The UCV radiators can be as described in U.S. Pat. Nos. 10,347,805; 10,074,784; 9,935,247 and 8,962,359 all herein incorporated by reference in their entireties to the extent that these references are not inconsistent with the present disclosure.

The use of the apparatus is targeted for airport security check points worldwide. These places will utilize this apparatus to sterilize the scanner security bins after a bin has been used during the TSA check point procedure.

Numerous other advantages and features of the present invention will be become readily apparent from the following detailed description of the invention and the embodiments thereof, and from the accompanying drawings.

### BRIEF DESCRIPTION OF THE DRAWINGS

In the following detailed portion of the present description, the teachings of the present application will be explained in more detail with reference to the example embodiment shown in the drawings, in which:

FIG. 1 is a schematic side view of an apparatus according to a first embodiment with portions removed to display underlying components.

FIG. 2A is a schematic front view of the apparatus of FIG. 1.

FIG. 2B is a schematic back view of the apparatus of FIG. 1.

FIG. 3 is a schematic plan view of the apparatus of FIG. 1 with portions removed to display underlying components.

FIG. 4 is a schematic front internal view of the apparatus of FIG. 1 with portions removed to display underlying components.

FIG. 5 is a schematic side internal view of the apparatus of FIG. 1 with portions removed to display underlying components.

### DETAILED DESCRIPTION

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

This application incorporates by reference U.S. Provisional Application 62/819,592 filed Apr. 15, 2019.

In the following detailed description, the apparatus according to the teaching of this application is in the form of a UVC security scanner bin sterilization apparatus, for sterilizing security bins.

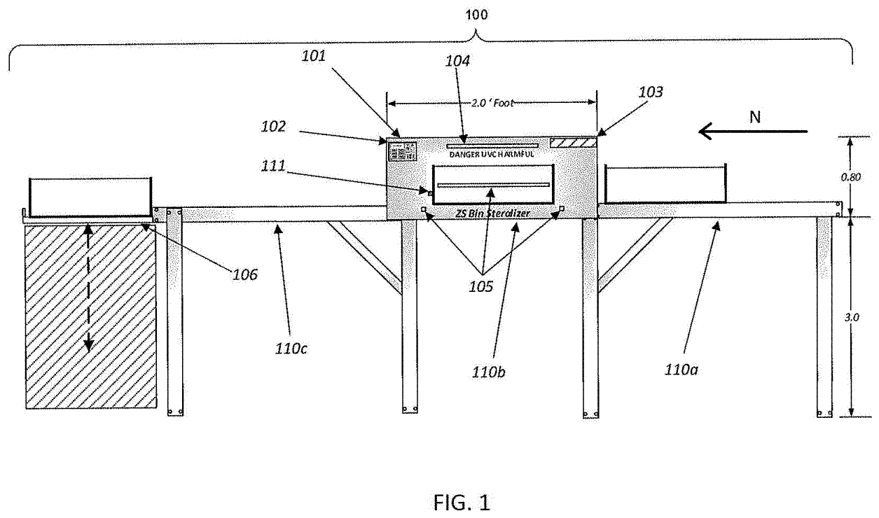

An embodiment of the UVC Security Scanner Bin Sterilization machine is illustrated in FIGS. 1-5. The apparatus 100 comprises an enclosure 101, conveyors 110a, 110b, 110c and a bin stacker 106. The enclosure 101 mounts a PLC/HMI 102 programmable logic controller PLC/HMI 102 with a human machine interface (PLC/HMI) that operates automatically or can be operated by manual control for overall machine operation and monitoring. The enclosure 101 also houses side UVC lights or UVC LED diode arrays 105a, 105b, front and back UVC lights or UVC LED diode arrays 105c, 105d and top UVC lights or UVC LED diode array 104, the center conveyor 110b and a bin sensor 111. The enclosure 101 also houses power supplies 103 to provide the UVC diode arrays or UVC lights, conveyors, stacker and the PLC/HMI, the necessary operating voltages.

The three conveyor belts move the security scanner bin in the direction “N” into the housing and into position for sterilization in the housing, and then out of the housing to the bin stacker. The first and second conveyors 110a, 110b operate at a preset speed while the third conveyor 110c operates at a faster speed for stacking purposes. The conveyors can be belt conveyors or powered roller conveyors which can contain a motor within one or more rollers, such as a POWERMOLLER 24 from Itoh Denki USA Inc.

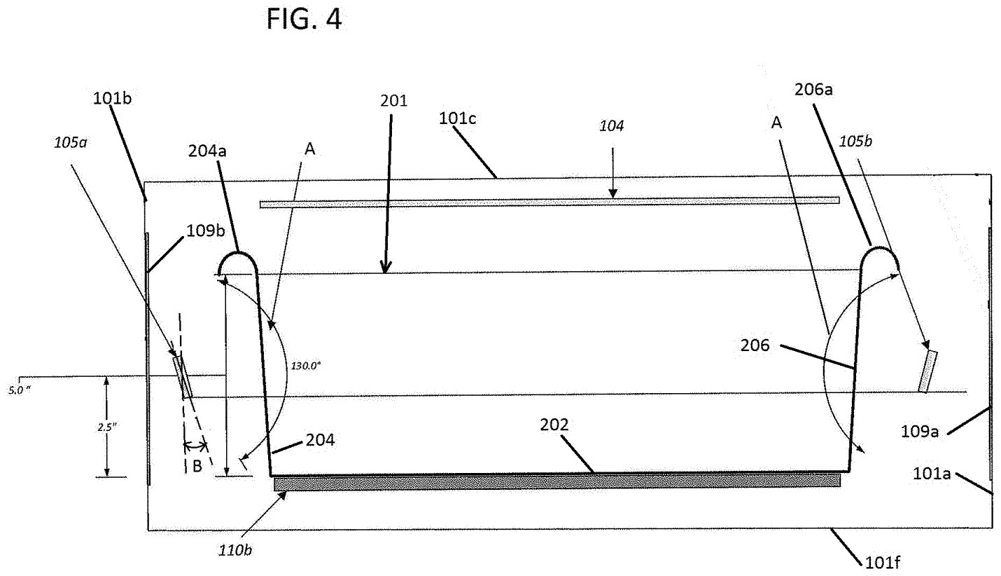

The LED lights or UVC LED diode arrays 105a, 105b are to the sides of the apparatus to radiate sides of the bin. The LED lights or UVC LED diode arrays 105c, 105d are adjacent to the ends of the apparatus to radiate the bottom, front and back of a bin 201 (FIG. 4). This UVC lights can radiate ultraviolet radiation at wavelengths less than 300 nm, or UVC LED diode arrays can radiate ultraviolet radiation in a wavelength range of 250 nm to 279 nm.

The housing includes sidewalls 101a, 101b, a top wall 101c, end walls 101d, 101e, and a bottom wall 101f. It is also possible that the conveyers are the bottom wall so a separate bottom wall is not necessary. The end wall 101d includes a front opening 101f and the end wall 101e includes a back opening 101g. The housing 101 and the internal components UVC lights or UVC LED diode arrays 105a, 105b, 105c, 105d and 104, and the center conveyor 110b are substantially symmetrical across a lateral and longitudinal center plane.

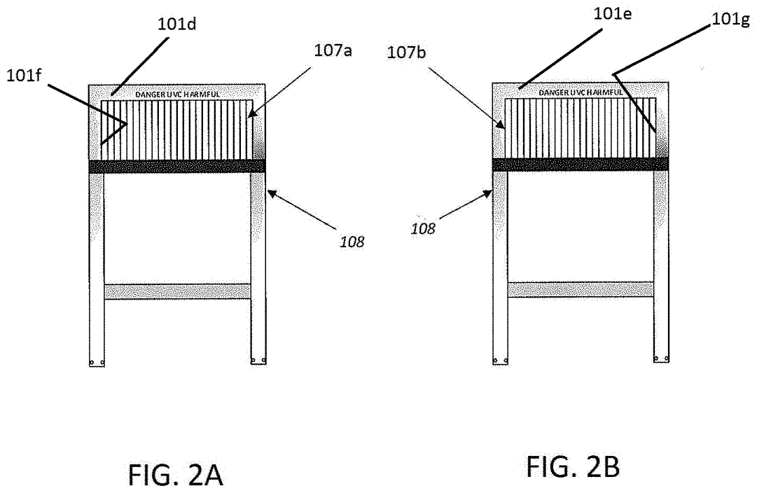

FIGS. 2A and 2B illustrate the UVC security scanner bin sterilization apparatus 100 having the end openings 101f, 101g of the enclosure 101 that are protected from UVC radiated leakage by PTFC coated curtains 107a, 107b.

The curtains 107a, 107b allow bins to pass into the housing 101 and out of the housing, while closing the openings 101f and 101g while the scanning is activated.

The apparatus 100 is supported by extruded aluminum supports 108.

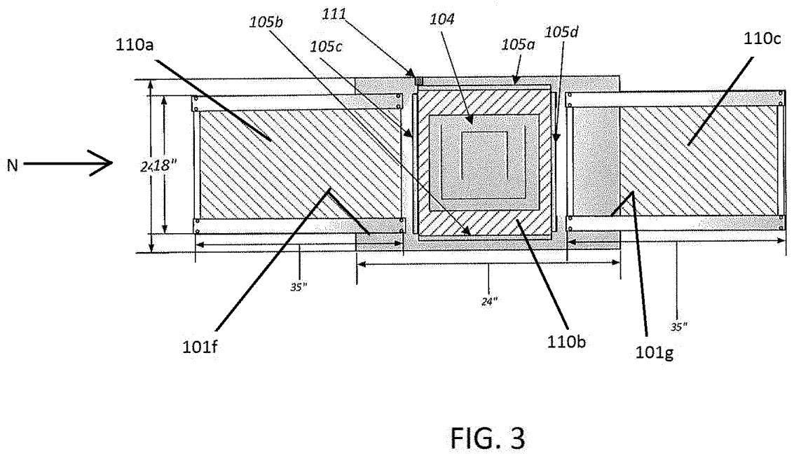

FIG. 3 shows a UVC lighting or UVC LED diode arrays 104, UVC lighting or UVC LED diode arrays 105a, 105b, 105c, 105d, bin sensor 111, conveyors 110a, 110b and 110c.

FIG. 4 illustrates a typical airport security scanner bin 201 includes a bottom wall 202, sidewalls 204, 206, end walls 208, 210. Each sidewall includes concave rim portions 204a, 206a on a top thereof. Each end wall includes concave rim portions 208a, 210a.

FIG. 4 illustrates the UVC lights or UVC LED diode arrays 105a and 105b, shown in an angular position with reference to the security scanner bin position. The internal reflective PTFE sheets 109a, 109b are shown mounted to an inner surface of the sidewalls 101a, 101b of the enclosure 101. The UVC light or UVC LED diode array 104 is shown in its mounted position. Also depicted is conveyor belt 110b.

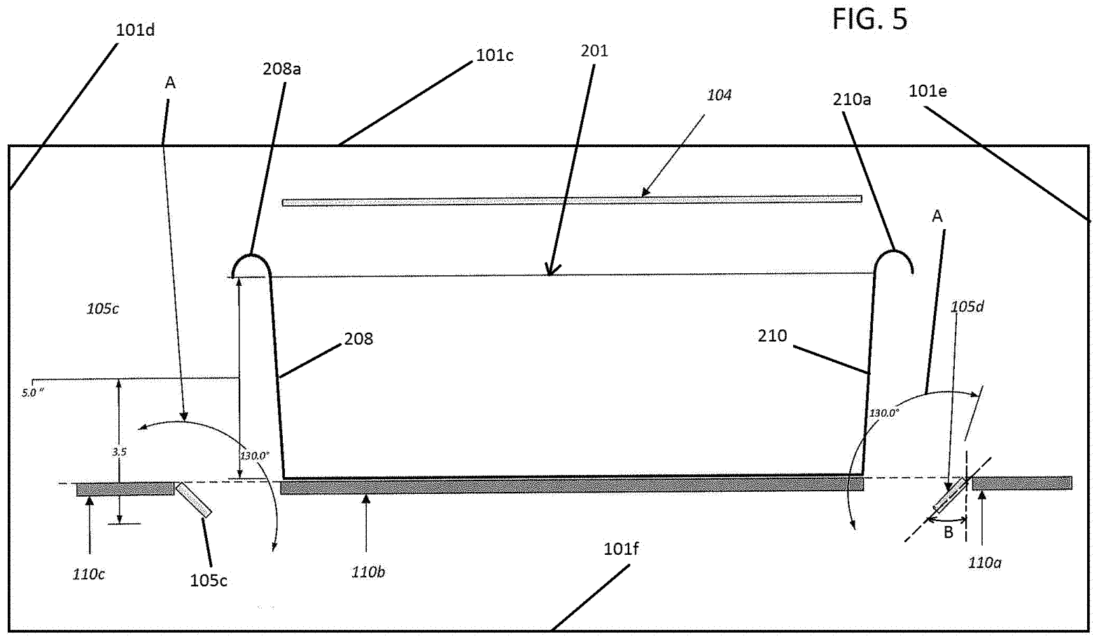

FIG. 5 illustrates the UVC lights or UVC LED diode arrays 105c and 105d in an angular position with reference to the security scanner bin position. The UVC light or UVC LED diode array 104 is shown in its mounted position. Also depicted are conveyor belts 110a, 110b and 110c.

The UVC light or UVC LED diode arrays 105a-105d can be inclined at an angle B from vertical within a range of about 30-45 degrees and have a radiation angle A of about 130 degrees. In this way, the UV light radiates over the sidewalls 204, 206 and end walls 208, 210 and also up into the insides and undersides of the concave rim portions 204a, 206a, 208a, 210a. The UVC light or UVC LED diode array 104 radiates on a top surface of the rim portions 204a, 206a, 208a, 210a and on the inside surfaces of the sidewalls 204, 206 and end walls 208, 210 and a top surface of the bottom wall 202. The reflective liner 109 enhances the radiation onto the bin.

In operation, once the conveyors 110a, 110b move the scanner bin 201 to the sensor 111, the conveyors stop, and the sterilization process activates. This sterilization process, radiating of UVC light, can operate for a programmed duration, between 3.5 to 5 seconds. Additionally, one of the front or back UVC radiators 105c, 105d, or both, can be operated while the bin is moving into or out of the housing to sterilize a bottom of the bin. The top UVC radiator 104 and the side UVC radiators 105a, 105b could be operated while the bin is moving through the housing as well.

From the foregoing, it will be observed that numerous variations and modifications may be incorporated without departing from the spirit and scope of the invention. It is to be understood that no limitation with respect to the specific apparatus illustrated herein is intended or should be inferred.

The invention claimed is:

1. An apparatus for sterilizing airport security scanner bins, comprising;
a housing having a housing bottom support, housing sidewalls, and a housing top wall, the housing having an open front housing end and an open back housing end;
the apparatus including a top UV radiator, and side UV radiators within the housing, and front and back UV radiators;
the housing and the open front and back ends sized to allow an airport security scanner bin to pass through the housing and inside the housing be radiated with UV light on a top, bottom, front wall, back wall and sidewalls of the bin.

2. The apparatus according to claim 1, wherein the housing bottom support comprises a conveyor.

3. The apparatus according to claim 1, wherein the front UV radiator is located adjacent the open front housing end and the back UV radiator is located adjacent to the open back housing end.

4. The apparatus according to claim 1, further comprising a bin stacking table for receiving bins from the open back housing end.

5. The apparatus according to claim 1, wherein the side UV radiators comprise UVC LED arrays.

6. The apparatus according to claim 1, wherein the top UV radiator comprises a UVC LED array.

7. The apparatus according to claim 1, wherein the front and back UV radiators comprise UVC LED arrays.

8. The apparatus according to claim 1, wherein the side UV radiators comprise UVC LED arrays having a length substantially equal to a length of the housing.

9. The apparatus according to claim 1, wherein the top UV radiator comprises a UVC LED array having a length substantially equal to a length of the housing.

10. The apparatus according to claim 1, wherein the front and back UV radiators comprise UVC LED arrays having a length substantially equal to a width of the housing.

11. The apparatus according to claim 1, further comprising a bin sensor for sensing that a bin is in position within the housing to turn on the UV radiators for a pre-selected period of time.

12. An apparatus for sterilizing airport security scanner bins, comprising;
a housing having a housing bottom conveyor, housing sidewalls, and a housing top wall, the housing having an open front housing end and an open back housing end;
the apparatus including a top UV radiator, side UV radiators within the housing, and front and back UV radiators;
the housing and the open front and back ends sized to allow an airport security scanner bin to pass through the housing while being radiated with UV light on a top, front wall, back wall and sidewalls of the bin.

13. The apparatus according to claim 12, wherein the front UV radiator is located adjacent the open front housing end and the back UV radiator is located adjacent to the open back housing end and are inclined to radiate UV light up into a concave portion of each bin end wall.

14. The apparatus according to claim 12, further comprising a bin stacking table for receiving bins from the open back housing end.

15. The apparatus according to claim 12, wherein the side UV radiators comprise UVC LED arrays and are inclined to radiate UV light up into concave portions each bin sidewall.

16. The apparatus according to claim 12, wherein the top UV radiator comprises a UVC LED array.

17. The apparatus according to claim 12, wherein the front and back UV radiators comprise UVC LED arrays.

18. The apparatus according to claim 122, wherein the side UV radiators comprise UVC LED arrays having a length substantially equal to a length of the housing.

19. The apparatus according to claim 12, wherein the top UV radiator comprises a UVC LED array having a length substantially equal to a length of the housing.

20. The apparatus according to claim 1, wherein the front and back UV radiators comprise UVC LED arrays having a length substantially equal to a width of the housing.
